(19) United States
(12) Patent Application Publication
(10) Pub. No.: US 2016/0235097 A1
(43) Pub. Date: Aug. 18, 2016

(54) FOOD COATING

(71) Applicants: Rousselot B.V., Son (NL); FORTIFIED FOOD COATINGS B.V., 's-Hertogenbosch (NL)

(72) Inventors: Diederik Johan Christofoor Bruins, 's-Hertogenbosch (NL); Paul Stevens, Gent Mendonk (BE)

(73) Assignee: Rousselot B.V., Son (NL)

(21) Appl. No.: 15/022,069

(22) PCT Filed: Sep. 15, 2014
(86) PCT No.: PCT/NL2014/050631

§ 371 Date: Mar. 15, 2016

(30) Foreign Application Priority Data
Sep. 16, 2013 (NL) 1040389

(51) Int. Cl. A23L 1/053; A23L 3/37; A61K 9/00

(52) U.S. Cl. CPC A23L 1/0052; A23L 3/37; A23L 1/0061; A23L 1/05625; A23L 1/09; A23L 1/296; A61K 9/0056; A23V 2002/00

(57) ABSTRACT

Described is a method for providing an outer surface of a foodstuff with a gel layer, comprising the steps of applying a liquid gelling composition comprising a gellable hydrocolloid and a plasticizer on the said outer surface of the foodstuff, and allowing the liquid gelling composition to gel while in contact with the outer surface of the foodstuff, therewith forming a gel layer directly covering the said outer surface of the foodstuff. Also described is a novel food coating composition, in particular for coating pre-prepared meals, that are to be heated before consumption. The gelling composition may also comprise additional substances such as nutrients, flavouring agents or medicaments. Also described is a food product coated with the said composition and a method for coating food stuff.

The invention relates to a method for providing an outer surface of a foodstuff, in particular pre-prepared meals, with a gel layer, to a gelling composition, to foodstuffs coated with the said composition, to a method for the preparation of the said composition, to a medicament or food supplement, included in the said composition and to a method for improving therapy compliance.

For consumers, in particular those, who do not prepare foodstuffs themselves, it is important that food stuffs remain fresh and have an appealing appearance during storage between the preparation of the foodstuff and consumption thereof. In the art, foodstuffs are usually covered by a thin plastic foil, such as Saran Wrap from Dow Chemical. However, such a foil allows space between the food stuff and the foil, and is not suitable to keep foodstuffs fresh when being frozen.

From US2004/0166205 it is known to lightly spray a 4% gelatin solution to blueberries and to cool these berries to allow the gelatin solution to gel to give these berries a ‘fresh’ appearance. However, the coated berries only have the ‘fresh’ appearance directly after being coated. The coating is weak, loses moisture, and berries cannot be kept at either ambient temperature or at refrigerator temperatures (4-10° C.) without deteriorating.

It has up to now not been possible to confer optimal fresh appearance and keepability to foodstuffs that are not directly consumed after preparation thereof. This is in particular a problem for kitchens and canteens that offer meals to consumers, which meals have been prepared earlier, or meals that are refrigerated or frozen before use, and need heating before consumption.

The present invention now obviates the above problem in that it provides a method for providing an outer surface of a foodstuff with a gel layer, comprising the steps of:

1) applying a liquid gelling composition comprising a gellable hydrocolloid and a plasticizer on the said outer surface of the foodstuff, 2) allowing the liquid gelling composition to gel while in contact with the outer surface of the foodstuff, therewith forming a gel layer directly covering the said outer surface of the foodstuff.

The term ‘gellable hydrocolloid’ means that the hydrocolloid in the said liquid gelling composition, which is preferably an aqueous medium such as a suitable solution or dispersion, is able to form a gel, in particular at room temperature. The skilled person is aware of suitable gellable hydrocolloids that can be used.

By incorporation of a plasticizer, it was found that the gel layer does not lose too much moisture during storage of the coated food stuff, and that a jelly coating with an attractive brilliant appearance is maintained, without significant drying of the gel layer and the food stuff. As a result, improved keepability as well as a more attractive appearance is obtained during storage. The skilled person is aware of suitable plasticizers that can be used.

The components of the said composition are preferably food grade, in order to allow the consumer to consume foodstuff coated with the said composition.

The term ‘directly covering’ means that the gel layer is in direct contact with the outer surface of the foodstuff, without leaving open spaces between the gel layer and the foodstuff, as would be the case when the foodstuff would be wrapped in an existing film, such as an edible film as described e.g. in WO2007/149276 or the above-described Saran Wrap.

It is to be understood that gelatin capsules, such as soft gelatin capsules and the preparation thereof, are not intended to be encompassed by the invention, as such capsules are not prepared by spraying a liquid solution onto a foodstuff. First of all, the contents of a soft gelatin capsule cannot be understood as a foodstuff. A foodstuff is herein intended to be a product, intended to be consumed in order to arrive, by the sole consumption of the foodstuff, at a satiety sensation, such as the sensation to have eaten, in particular to have eaten a meal. A medicament or food additive as such is not a foodstuff as intended herein, as these are not intended to give a satiety sensation, but to improve health. Secondly, soft gelatin capsules are not produced by applying a liquid gelling composition to the outer surface of a foodstuff, as in the process of preparation of soft gelation capsules, a gelation solution is firstly gelled to form gelled gelatin sheets, that are used as material for capsule walls which are melted together after being filled with e.g. a medicament.

As the composition of the invention is liquid, it is pumpable in order to allow optimal contact and sealing of the food stuff by the coating composition upon application thereof. To this end, step 1) preferably comprises casting, spraying or atomizing the liquid gelling composition onto the foodstuff.

Preferably, the liquid gelling composition is evenly applied to the outer surface of the foodstuff, therewith providing a continuous gel layer on the foodstuff, giving the foodstuff optimal appearance and protection.

In a preferred embodiment, the gel layer formed in step 2) adheres to the said outer surface of the foodstuff. This means that the gel layer, after application of the gelling composition and gelling thereof on the outer surface of the foodstuff, sticks to foodstuff without getting loose thereform. Again, an optimal protection of the foodstuff is obtained.

The gelling composition is preferably applied with a thickness of 0.4 mm-1.6 mm, more preferably of 0.6-1.2 mm or 0.7-1.2 mm, and can be applied in a continuous manner. The resulting coating, or gel layer, will have the same thickness, without imparting on the taste and mouth feel.

The weight of the gel layer is preferably between 1 and 30% of the total weight of the foodstuff including the gel layer, preferably between 2 and 20%, more preferably between 3 and 15%, most preferably between 4 and 10%. A meal to be coated with a gel layer according to the method of the invention has usually a weight of about 100 up to about 600 g, preferably of about 350 to 450 g, a usual weight of a normal meal that results in a satiety sensation after consumption. For such a meal, about 20 to 40 g of gelling composition is preferably used for covering the said meal. The said meal is preferably present on a substantially flat plate or in a rectangular or bowl shaped open container, the gelling composition preferably being applied to the foodstuff such, that substantially the complete foodstuff on the plate or in the container is sealed from the air, in order to confer optimal keepability and storage quality to the foodstuff while keeping optimal appearance. To this end, the gelling composition is preferably also applied on the plate or container, at least at the edges of the foodstuff, to provide a continuous gel layer starting on the plate or container surface, covering and therewith sealing the complete foodstuff. However, the gelling composition can also be applied only on one or more portions of the meal, and accordingly, less of the gelling composition is needed.

The gelling composition preferably gels at ambient temperature, so that a gel layer can conveniently be prepared. If the foodstuff is intended to be frozen until use, a gelling composition can be chosen that does not gel at ambient temperature but e.g. at refrigerator temperature. In the latter case, the gel layer would spontaneously liquefy when the food stuff is taken from the freezer and kept at ambient temperature, resulting in an attractive edible foodstuff. It is to be understood that a composition that gels at ambient temperature also gels at lower temperatures. The skilled person is very well capable of choosing the proper hydrocolloid to arrive at the envisaged gelling temperature of the gelling composition.

The gelling composition preferably liquefies at a temperature above 40° C., preferably above 50° C., more preferably above 60° C. At ambient temperatures, the gel layer will remain intact and provides optimal protection. Upon heating the meal to an envisaged consumption temperature, e.g. 60° C. or 70° C., the gel coat of the foodstuff will melt, i.e. liquefy, giving the meal optimal appearance, while the meal is still fresh after storage. For the same reason, the gelling composition preferably liquefies at a temperature below 90° C., preferably below 80° C., more preferably below 70° C. The gelling composition is preferably chosen such, that the composition gels at ambient temperature whereas the obtained gel layer preferably melts at or below the temperature at which the foodstuff is heated for consumption. In such a case, the coating is liquid at the consuming temperature, and therefore, at that temperature, the coating will be disappeared, providing optimal appearance, mouth feel and taste sensation to the consumer without any jelly mouth feel.

The gellable hydrocolloid is preferably chosen from the group, consisting of gelatine, pectin, agar, carrageenan and gum Arabic. The skilled person will be aware of suitable alternative hydrocolloids. In particular, gelatine has been shown as a very suitable hydrocolloid for the composition of the invention. Gelatine is a mixture of water-soluble proteins, derived from collagen. Gelatine is obtained e.g. by partial hydrolysis of collagen, obtained by aqueous extraction of skin, tendons, ligaments, bones etc. in acid or alkali conditions, or by enzymatic hydrolysis. Gelatine obtained by acid treatment is called Type A gelatine, whereas Type B gelatine is derived from alkali treatment. Gelatine is commonly used as gelling agent in food, pharmaceuticals and cosmetics.

Gellable hydrocolloids also have a nutritional contribution to the foodstuff and have a positive effect on satiety for the consumer. Therefore, by incorporating a gellable hydrocolloid, in particular gelatin, the foodstuff may contain accordingly less carbohydrates and/or fats, resulting in a foodstuff or meal having less calories, but result in the same or similar satisfaction of satiety for the consumer, which may be a powerful tool against obesitas. Also, the foodstuff or meal prepared according to the present invention, i.e. comprising a gelatin coating, will have an elevated protein level, and consumption of such a coated foodstuff or meal may therefore be used against malnutrition.

A clear, elastic and tacky gel is formed by gelatine, allowing an optimal gel to form on and around the food stuff having good adhering properties. In particular when the food stuff, such as a complete or partial meal, is provided on a plate or bowl, a composition comprising gelatine has shown to form a gel around the food and forms a continuous layer over the food and the plate, while adhering to both the food and the plate. The said gel layer not only confines the food on the plate, but also fixes the food thereon, so that the food will not, or less often, slide from the plate when the plate is tilted during handling.

Gelatine is defined by its bloom, or gel strength. It forms a thermoreversible gel at room temperature, and dissolves in hot water. it is e.g. used as gel former and texturizer in fruit gums and gelatine desserts. The gel strength of a gelatine gel can be determined by standardized apparatuses (GME, Brussels, Belgium), such as a QTS 25 Texture Analyser (Brookfield Viscometers) or a Texture Analyser TA-XT2 (Stable Micro Systems Ltd., London, United Kingdom), and is indicated by a bloom number (also referred herein as ‘bloom value’). The test was originally developed in 1925 by O. T. Bloom (U.S. Pat. No. 1,540,979 and U.S. Pat. No. 2,119,699). The test determines the weight (in grams) needed by a probe (normally with a diameter of 0.5 inch) to deflect the surface of the gel 4 mm without breaking it. The result is expressed in bloom (grades). It is usually between 30 and 300 bloom. The higher the bloom number, the stronger the gel. To perform the bloom test on gelatine, a 6.67% gelatine solution is kept for 17-18 hours at 10° C. prior to being tested. The term low bloom' reflects a bloom number of 50-125, whereas ‘medium bloom’ reflects a bloom number of 150-250, and ‘high bloom’ reflects a bloom number of 250-325.

Further hydrolysis results in so-called ‘hydrolysed gelatine’, also known as ‘hydrolysed collagen’, a peptide preparation having an average molecular weight of less than 20 kDa, e.g. between 500 Da and 15 kDa. The molecular weight of the peptides in hydrolysed gelatine is preferably between 100 and 5000 Da, more preferably between 500 and 5000 Da. Because of the relatively small molecules, hydrolysed gelatine has no jellifying effect. Hydrolysed collagen is e.g. used as texture conditioner and moisturizer in topical crèmes, and is also used in nutritional products because of the high glycine, proline and hydroxyplroline content.

The average molecular weight of both gelatine and hydrolysed gelatine can be determined by methods, known in the art, e.g. by HPLC size exclusion chromatography.

The skilled person can chose any known gelatine for the envisaged aim. In case halal or kosher food is to be coated, the choice will preferably be gelatine of bovine or fish origin. Also, if fish is to be coated, gelatine of fish origin may be contemplated. Porcine gelatine can however be used as well.

An increase in bloom, and/or the gelatine content, will increase the viscosity of the composition of the present invention. The skilled person will be capable of choosing the proper gelatine and content for the envisaged aim. Accordingly, the gelatine preferably has a bloom value of 100-300, rendering the composition suitable to be industrially processed.

The composition according to the invention preferably comprises at least 10 w/w %, and preferably 30 or less w/w %, based on the total weight of the composition, gellable hydrocolloid, in particular gelatin. The weight is including the water content of the materials. For example, gelatin may have a water content of 12 w/w %.

When the amount of the components of the composition is defined based on dry weight, i.e. excluding any water content in the composition and the components thereof, the gelling composition preferably comprises at least 25 w/w %, preferably at least 30 w/w %, more preferably at least 35 w/w % and most preferably at least 40 w/w %, based on the dry weight of the composition, gellable hydrocolloid.

The plasticizer preferably comprises a polyol, preferably glycerol or sorbitol or a combination thereof, and most preferably comprises glycerol.

The gelling composition preferably comprises at least 2 w/w %, and preferably 15 or less w/w %, based on the total weight of the composition, plasticizer.

The gelling composition is suitable for coating food stuffs, such as partial or complete meals, that have been pre-prepared, and are stored for a period of time before consumption. The coating provides a protection layer, confers a brilliant attractive appearance and moisture loss from the food stuff is avoided. As will be discussed later on, the composition is also very suitable as carrier for additional substances such as food supplements and pharmaceuticals.

In case the food stuff or meal is to be frozen for storage, the composition of the invention preferably comprises a cryoscopic agent, i.e. an agent effective in lowering the freezing point. By incorporation of a cryoscopic agent, the formation of ice crystals takes place at a much lesser degree or is prevented. The skilled person is aware of suitable cryoscopic agents. The composition preferably comprises at least 1 w/w %, more preferably at least 2 w/w %, and preferably 10 w/w % or less, more preferably 7 w/w % or less, based on the total weight of the composition, of the cryoscopic agent. Above the said range, some colouring may appear in the coating which is less attractive. Any food grade cryoscopic agent will contribute to the limitation of ice crystal formation, but in the composition of the invention, the cryoscopic agent preferably comprises a mono- or disaccharide or a combination of two or more thereof. The mono- or disaccharide is preferably chosen from the group, consisting of glucose, fructose, sucrose, maltose and lactose, or a combination of two or more thereof. The cryoscopic agent preferably comprises fructose. The preferred cryoscopic agents confer an envisaged freezing point depression, resulting in less ice crystal formation. As less or no ice crystals are formed, the appearance of the food stuff during and after freezing remains very attractive. This is very advantageous e.g. when presenting frozen food stuff, like frozen pizza's in a refrigerated display. Such food stuffs have a very fresh and attractive appearance.

In order to further limit the ice crystal formation, the composition preferably comprises peptides having a molecular weight of up to 20 kDa, in particular peptides known in the art as ‘AFP’, i.e. Anti Freeze Peptides, and preferably have a molecular weight of up to 5 kDa. Such peptides contribute to the freeze point depression and counteract ice crystal growth, by forming a film around small crystals. By incorporation of peptides, a coating can be formed on food stuffs that has even less or smaller ice crystals. Further, peptides have a nutritional contribution and can accordingly be used against malnutrition. Further, peptides have, like the gellable hydrocolloid, in particular gelatin, a positive effect on satiety. By incorporating peptides, less carbohydrates and/or fats need to be present in the foodstuff, resulting in a foodstuff or meal having less calories, but result in the same or similar satisfaction of satiety for the consumer.

In a preferred embodiment, the peptides are derived from hydrolysed gelatine. Therefore, the composition preferably comprises hydrolysed gelatine. Hydrolysed gelatine is an optimal source for food grade peptides, suitable for the composition of the invention. Hydrolysed gelatine is particularly suitable in view of the above described satiety effect. When hydrolysed gelatine is incorporated in the gelling composition, a low calorie meal can be prepared that still converts a satiety sensation to the consumer. The molecular weight of the peptides is preferably between 100 and 5000 Da, more preferably between 500 and 5000 Da.

The gelling composition preferably comprises at least 5 w/w %, and preferably 15 w/w % or less, based on the total weight of the composition, peptides, preferably hydrolyzed gelatin. However, the skilled person will be aware of other suitable peptides from other sources. Suitable hydrolysed gelatine is e.g. Peptan of Rousselot B.V., The Netherlands.

Preferably, the gelling composition comprises an anti-foaming agent, to avoid foam formation during coating. The anti-foaming agent can be any food grade agent known in the art, but it is preferred for the anti-foam agent to comprise hydrophylic C8-C10 mono-, di- and triglycerides, such as caprylic/capric glycerides produced by Sasol (Germany), under the trade name Witafrol 7420. The gelling composition preferably comprises 10 -1000 ppm, more preferably 20-500 ppm, most preferably 20-40 ppm anti-foaming agent.

Apart from the contribution to the satiety effect and the replacement possibilities for carbohydrates and/or fats in the foodstuff by the gelling composition, in particular gelatin and optionally hydrolysed gelatin, the coating composition is very well suited as a carrier or vehicle for such additional substances, in order to supplement the food stuff or meal with envisaged substances. The gelling composition therefore preferably comprises one or more additional substances, chosen from the group, consisting of food supplements, such as nutrients, vitamins, calcium, trace elements, neutraceuticals, antioxidants; flavouring agents such as taste improvers; odorants; colorants; and pharmaceuticals. Said additional substances are conveniently added to the food, without the need or separate ingestion of said substance in the form of pills etcetera. So the coating composition can be used e.g. in hospitals, old people's homes, health resorts etc. Coating compositions can also be tailored to individual patients to comprise the exact medication prescribed for the said patient so that the said patient will ingest his or her medicine by just eating the meal, improving the patients compliance significantly.

For example commercially available nutrient mixes can be incorporated in the gelling composition that improve or sustain the health of the consumer, such as blood cholesterol lowering, prophylaxis of stroke and heart disease, or is otherwise beneficial to the consumer, such as for enhancement of mental energy, e.g. against fatigue, improvement of enthusiasm and attention. For example, vitamin mixes such as of the Qual series (DSM, The Netherlands), nutritional lipids such as life'sDHA and life's AHA (DSM, The Netherlands) and nutroceutical mixes such as Oatwell, Fruitflow, ALL-Q, FloraGlo, PeptoPro, resVida (DSM, The Netherlands) can be incorporated in the gelling composition. Further, for diabetes patients, meals can be prepared wherein in the gel layer a suitable amount of one or more blood glucose regulating medicaments are incorporated, that may result in obviating the need for the patient to inject the insulin. The amount of such medicaments in the gel layer can be fit in an exact manner to the need of the patient, e.g. depending on the amount of carbohydrates in the coated meal. Examples of suitable blood glucose regulating medicaments are e.g. a-glucosidase inhibitors (such as acarbose), biguanides (such as metformine), dipeptidylpeptidase IV inhibitors (such as linagliptine, saxagliptine, sitagliptine and vildagliptine), meglitinides (such as repaginide), sulfonyl urea derivatives (such as tolbutamide, glicazide, glibenclamide, glimepride), thiazolidinediones (such as pioglitazon) and incretin mimetics (such as exenatide and liraglutide). When a medicament against Alzheimer's disease (such as galantamine, rivastigmine and memantine) is incorporated in the coating composition of the invention, Alzheimer patients that consume a meal coated with such a coating of the invention will automatically get their medication, without the risk of forgetting to take the medication.

In another embodiment of the method of the invention, the method further comprises providing a second gel layer onto the foodstuff, the composition of the second gel layer preferably differing from that of the first gel layer, in particular in presence and/or composition of one or more additional substances.

A second gel layer can be applied onto the food stuff before, or after the above-described first gel layer. In this way, a bilayered or multi-layered structure is obtained, wherein the compositions of the first and second layer may be different from one another. Preferably, both the first and second layer are according to the invention, although this is not a requisite. A second layer can be applied, e.g. in order to increase the strength and transparency of the coating. However, a monolayer, obtained by coating a food stuff with the composition of the invention, is preferably transparent as well. In case a bilayer is to be prepared, both layers can be thin, i.e. in the range of 0.4-0.5 mm, although other thicknesses are possible. The outer layer can be thinner than the inner layer, or vice versa. As ice crystals tend to form on the outer layer, the content of cryogenic agent is preferably higher in the outer layer, whereas the inner layer may preferably be more rigid, by having a higher gelatine content, or the gelatine of the inner layer can be of a higher bloom value.

The invention also relates to a gelling composition comprising, based on the total weight of the composition 12-25 w/w %, preferably 16-20 w/w % gellable hydrocolloids, 5-15 w/w %, preferably 7.5-12.5 w/w % peptides having a molecular weight of up to 20 kDa, preferably up to 5 kDa, 2-12 w/w %, preferably 4-8 w/w % plasticizer, 1-10 w/w %, preferably 3-7 w/w % cryoscopic agent, and optionally 10-1000 ppm, preferably 20-40 ppm anti-foaming agent and 0.5-15 w/w %, preferably 2-4 w/w % additional substances. Preferences of these ingredients are discussed above.

In a particular embodiment, the gelling composition comprises 12-25 w/w %, preferably 16 -20 w/w % gelatine, 5-15 w/w %, preferably 7.5-12.5 w/w % hydrolysed gelatine, 2-12 w/w %, preferably 4-8 w/w % glycerol, 1- 10 w/w %, preferably 3-7 w/w % fructose, and optionally 10-1000 ppm, preferably 20 -40 ppm anti-foaming agent and 0.5-15 w/w %, preferably 2-4 w/w % additional substances.

A gelling composition having the above ingredients in the above ranges provide for a very suitable coating, conferring a brilliant appearance to the food stuff, even while frozen, confers food stability, and maintains taste and texture. Further, taste improvers and other additional agents can improve the food quality and/or the nutritional value thereof.

The gelling composition of the invention is preferably for coating a pre-prepared food stuff or meal, which food stuff or meal is preferably to be heated before consumption. By heating the coated meal or foodstuff, the coating will melt, and will depending on the food, penetrate the food. The consumer will not have the experience of eating a coated food stuff, but a fresh food instead.

The invention further relates to a foodstuff, in particular a pre-prepared meal, comprising, adhered to the outer surface thereof, a gel layer of the gelling composition of the invention.

The foodstuff is preferably a ready-to-consume warm meal, and is preferably prepared well before consumption, i.e. needs storage. Storage can take place at ambient temperature, i.e. at 16 -24° C., preferably 18-20° C., or in a refrigerator (i.e. at a temperature of 4-10° C., preferably 6-8° C.), or in a freezer at freezing conditions (i.e. at a temperature of below 0° C., preferably between -5° C. and −25° C., more preferably between −12° C. and −20° C.). The foodstuff is therefore preferably stored until consumption under freezing conditions, under refrigerator conditions or at ambient temperature, and preferably intended to be heated for consumption to at least 50° C. In particular, such a pre-prepared meal can be a frozen meal to be heated, or a meal, intended to be heated by the use of a microwave oven.

The pre-prepared meal is preferably a microwave or frozen meal, although it is very well possible to provide a meal according to the invention, prepared by a kitchen or canteen of a large institution, such as a university of hospital, or hotels or restaurants, where the meals are prepared and then allowed to cool e.g. to ambient or refrigerator temperatures, and are reheated just before consumption.

By the said heating, the gel layer will melt without any negative impact on mouth feel and taste sensation. On the contrary, the foodstuff, in particular in the case of frozen pizza coated with a gel layer according to the invention, even tastes better than a similar pizza, kept at similar conditions without being coated.

As discussed above, the gel layer is preferably a continuous gel layer, has a thickness of preferably 0.4 mm-1.6 mm, more preferably of 0.6-1.2 mm, or even 0.7-1.2 mm, and gels at ambient temperature. The gelling composition of the foodstuff preferably liquefies at a temperature above 40° C., more preferably above 50° C., even more preferably above 60° C., and preferably at a temperature below 90° C., more preferably below 80° C., even more preferably below 70° C.

The invention further relates to a method for the preparation of a coated food stuff, comprising the application of the composition of the invention onto the food stuff.

The gelling composition can be applied to the food stuff at a temperature where the composition gels, e.g. at room temperature or at lower temperatures. The composition can also be applied onto frozen food stuffs, or the food stuff, coated at e.g. room temperature can be refrigerated to e.g. 4° C. or a freezing temperature of e.g. -20° C. The invention also relates to a method for the preparation of a liquid gelling composition of the invention, comprising the steps of:
a) providing an aqueous medium,
b) adding the plasticizer to the aqueous medium,
c) adding the gellable hydrocolloid to the aqueous medium,
d) mixing to a homogenous mixture,
e) allowing the mixture of step d) to gel,
f) melting the gelled composition of step e) providing the liquid gelling composition.

The aqueous medium can be water, or a solution or dispersion comprising one or more of the envisaged ingredients, such as one or more of the additional substances such as nutrients. The liquid gelling composition may also be a viscous mass, as long as it is pumpable and instantly flowing. E.g. a low viscous mass may still be suitable for casting purposes. The melting step usually comprises heating the mixture to above the temperature where the hydrocolloid, in particular gelatin liquefies, resulting in a liquid, or at least instantly flowing mass.

The invention also relates to a medicament or pharmaceutical or a food supplement, incorporated in a gelling composition of the invention.

In another embodiment, the invention relates to a method for improving the therapy compliance of a patient or patient group, comprising providing a food stuff or meal to the said patient or patient group, the said food stuff or meal being coated with the gelling composition of the invention, the composition comprising one or more pharmaceuticals for the said therapy. The food stuff or meal is preferably heated before providing the said food stuff or meal to the patient or patient group.

In still another embodiment, the invention relates to a method of administration of one or more of food supplements, such as nutrients, vitamins, calcium, trace elements, neutraceuticals, antioxidants, and/or pharmaceuticals to a subject, comprising the step of allowing the subject to ingest a food stuff, the food stuff being coated with a gelling composition of the invention, the composition comprising the one or more food supplements and/or pharmaceuticals.

The food stuff is preferably heated before allowing the subject to ingest the said food stuff.

The invention is now further explained by way of the following examples which are not intended to limit the scope of the invention. The calculated weight percentages in the tables below are given in two decimals, so the total percentage can differ slightly from the total of 100.00 for that reason.

### EXAMPLE 1

### Nutrient Enriched Food Coating

The following ingredients were used to prepare a coating composition:

TABLE 1

| Ingredients: | Amount (g) | Wt. % |
|---|---|---|
| Water | 1701.98 | 56.73 |
| Gelatine (PS250 Rousselot) | 541.32 | 18.04 |
| Hydrolysed gelatine | 318.42 | 10.61 |
| (Peptan ® P2000 LD Rousselot) | | |
| Glycerol (AMI) | 178.32 | 5.94 |
| Fructose (UNIVAR) | 149.66 | 5.00 |
| Nutrients (Premix NL37890 DSM) | 70.49 | 2.35 |
| Butarome ® (Buter Essence, NL) | 39.80 | 1.33 |
| TOTAL | 3000 | 100 |

The ingredients were added in the water and mixed until a homogenous solution was obtained.

The homogenous mixture was allowed to swell (i.e. gellify) at room temperature during 1.5 hours. Subsequently, the gelled mixture was kept at 60° C. until the mixture was melted, for about 45 minutes, and allowed to stand for 10 minutes. The mixture was used to cast over pre-prepared meals having room temperature, comprising boiled potatoes, boiled peas and carrots as well as a Wiener Schnitzel. The same was done by spraying the composition over the meal, using a spray nozzle. The thickness of the coating was about 0.9 mm. The meals were stored at 4° C. and at -20° C.

After 24, 36, 48 and 72 hours of storage at these temperatures, the meals had an attractive brilliant appearance, and only after 72 hours at -20° C., minor formation of ice crystals was observed. No taste loss or deterioration of texture was observed in any of the meals.

Similar gelling compositions were made differing from the above composition in the choice of nutrients. E.g. similar mixes were prepared with equal amounts of several nutritional supplements of DSM, the Netherlands, such as those given above.

### EXAMPLE 2

### Food Coating for Meals for Lowering Risk of Heart Disease and Stroke

The following ingredients were used to prepare a coating composition:

TABLE 2

| Ingredients: | Amount (g) | Wt. % |
|---|---|---|
| Water | 1701.98 | 56.73 |
| Gelatine (PS250 Rousselot) | 541.32 | 18.04 |
| Hydrolysed gelatine | 318.42 | 10.61 |
| (Peptan ® P2000 LD Rousselot) | | |
| Glycerol (AMI) | 178.32 | 5.94 |
| Fructose (UNIVAR) | 107.88 | 3.60 |
| Fruitflow ® (DSM, The Netherlands) | 45.59 | 1.52 |
| resVida ® (DSM, The Netherlands) | 10.89 | 0.36 |
| Nutrients (Premix NL37890 DSM) | 55.79 | 1.86 |
| Butarome ® (Buter Essence, NL) | 39.80 | 1.33 |
| TOTAL | 3000 | 100 |

The meals were prepared as described for example 1, stored at 4° C. for 24 hours and to be given to elderly people in a retirement institution.

### EXAMPLE 3

### Food Coating for Meals for Lowering LDL Cholesterol Level in the Blood

The following ingredients were used to prepare a coating composition:

TABLE 3

| Ingredients: | Amount (g) | Wt. % |
|---|---|---|
| Water | 1701.98 | 56.73 |
| Gelatine (PS250 Rousselot) | 541.32 | 18.04 |
| Hydrolysed gelatine | 318.42 | 10.61 |
| (Peptan ® P2000 LD Rousselot) | | |
| Glycerol (AMI) | 178.32 | 5.94 |
| Fructose (UNIVAR) | 107.88 | 3.60 |
| Oatwell ® (DSM, The Netherlands) | 40.59 | 1.35 |
| resVida ® (DSM, The Netherlands) | 15.89 | 0.53 |
| Nutrients (Premix NL37890 DSM) | 55.79 | 1.88 |
| Butarome ® (Buter Essence, NL) | 39.81 | 1.33 |
| TOTAL | 3000 | 100 |

The meals were prepared as described for example 1, stored at 4° C. for 24 hours and to be given daily to patients having an elevated blood LDL cholesterol level of above 4.5 mmol/l. Over time, LDL cholesterol level decreased in the majority of the patients to a level of 3.6-4.1 mmol/l.

### EXAMPLE 4

### Food Coating for Meals Against Fatigue, Increase of Enthusiasm, Attention and Mental Energy

The following ingredients were used to prepare a coating composition:

TABLE 4

| Ingredients: | Amount (g) | Wt %. |
|---|---|---|
| Water | 1701.98 | 56.71 |
| Gelatine (PS250 Rousselot) | 541.32 | 18.04 |
| Hydrolysed gelatine | 318.42 | 10.61 |
| (Peptan ® P2000 LD Rousselot) | | |
| Glycerol (AMI) | 178.32 | 5.94 |
| Fructose (UNIVAR) | 107.88 | 3.60 |
| Fruitflow ® (DSM, The Netherlands) | 25.59 | 0.87 |
| resVida ® (DSM, The Netherlands) | 30.89 | 1.03 |
| Nutrients (Premix NL37890 DSM) | 55.79 | 1.86 |
| Butarome ® (Buter Essence, NL) | 39.81 | 1.33 |
| TOTAL | 3000 | 100 |

The meals were prepared as described for example 1, stored at 4° C. for 24 hours and to be given to elderly people in a retirement institution. It was observed by nursery staff that after a week of daily consumption of such coated meals, the elderly people appeared to be more vivid and happier.

### EXAMPLE 5

### Food Coating for Meals for Diabetes Patients

The following ingredients were used to prepare a coating composition:

TABLE 5

| Ingredients: | Amount (g) | Wt. % |
|---|---|---|
| Water | 1701.98 | 56.71 |
| Gelatine (PS250 Rousselot) | 541.32 | 18.04 |
| Hydrolysed gelatine | 318.42 | 10.61 |
| (Peptan ® P2000 LD Rousselot) | | |
| Glycerol (AMI) | 178.32 | 5.94 |
| Fructose (UNIVAR) | 107.88 | 3.60 |
| Acarbose (Glucobay - Bayer, | 2.59 | 0.09 |
| Germany) | | |
| Nutrients (Premix NL43432 DSM) | 30.89 | 1.03 |
| Nutrients (Premix NL37890 DSM) | 70.49 | 2.35 |
| Butarome ® (Buter Essence, NL) | 39.81 | 1.33 |
| TOTAL | 3000 | 100 |

### EXAMPLE 6

### Food Coating for Meals for Alzheimer Patients

The following ingredients were used to prepare a coating composition:

TABLE 6

| Ingredients: | Amount (g) | Wt. % |
|---|---|---|
| Water | 1701.98 | 56.71 |
| Gelatine (PS250 Rousselot) | 541.32 | 18.04 |
| Hydrolysed gelatine | 318.42 | 10.61 |
| (Peptan ® P2000 LD Rousselot) | | |
| Glycerol (AMI) | 178.32 | 5.94 |
| Fructose (UNIVAR) | 108.21 | 3.61 |
| Nemtadine (Actavis - Netherlands) | 2.59 | 0.01 |
| Nutrients (Premix NL43432 DSM) | 30.89 | 1.03 |
| Nutrients (Premix NL37890 DSM) | 80.75 | 2.69 |
| Butarome ® (Buter Essence, NL) | 39.81 | 1.33 |
| TOTAL | 3000 | 100 |

1. Method for providing an outer surface of a foodstuff with a gel layer, comprising the steps of:
1) applying a liquid gelling composition comprising a gellable hydrocolloid and a plasticizer on the said outer surface of the foodstuff,
2) allowing the liquid gelling composition to gel while in contact with the outer surface of the foodstuff, therewith forming a gel layer directly covering the said outer surface of the foodstuff.

2. Method of claim 1, wherein step 1) comprises casting, spraying or atomizing the liquid gelling composition onto the foodstuff.

3. Method of claim 1 or 2, wherein the liquid gelling composition is evenly applied to the outer surface of the foodstuff, therewith providing a continuous gel layer on the foodstuff.

4. Method of any of the preceding claims, wherein the gel layer formed in step 2) adheres to the said outer surface of the foodstuff.

5. Method of any of the preceding claims, wherein the gelling composition is applied with a thickness of 0.4mm -1.6 mm, preferably of 0.6-1.2 mm.

6. Method of any of the preceding claims, wherein the weight of the gel layer is between 1 and 30% of the total weight of the foodstuff including the gel layer, preferably between 2 and 20%, more preferably between 3 and 15%, most preferably between 4 and 10%.

7. Method of any of the preceding claims, wherein the gelling composition gels at ambient temperature.

8. Method of any of the preceding claims, wherein the gelling composition liquefies at a temperature above 40° C., preferably above 50° C., more preferably above 60° C.

9. Method of any of the preceding claims, wherein the gelling composition liquefies at a temperature below 90° C., preferably below 80° C., more preferably below 70° C.

10. Method of any of the preceding claims, wherein the gellable hydrocolloid is chosen from the group, consisting of gelatin, pectin, agar, carrageen and gum Arabic.

11. Method of claim 10, wherein the gellable hydrocolloid comprises gelatin.

12. Method of claim 11, wherein the gelatin has a bloom value of 100-300.

13. Method of any of the preceding claims, wherein the gelling composition comprises at least 10 w/w %, based on the total weight of the composition, gellable hydrocolloid.

14. Method of any of the preceding claims, wherein the gelling composition comprises 30 or less w/w %, based on the total weight of the composition, gellable hydrocolloid.

15. Method of any of the preceding claims, wherein the gelling composition comprises at least 25 w/w %, preferably at least 30 w/w %, more preferably at least 35 w/w % and most preferably at least 40 w/w %, based on the dry weight of the composition, gellable hydrocolloid.

16. Method of any of the preceding claims, wherein the plasticizer comprises a polyol.

17. Method of claim 16, wherein the plasticizer comprises glycerol or sorbitol or a combination thereof.

18. Method of claim 17, wherein the plasticizer comprises glycerol.

19. Method of any of the preceding claims, wherein the gelling composition comprises at least 2 w/w %, based on the total weight of the composition, plasticizer.

20. Method of any of the preceding claims, wherein the gelling composition comprises 15 or less w/w %, based on the total weight of the composition, plasticizer.

21. Method of any of the preceding claims, wherein the gelling composition comprises a cryoscopic agent.

22. Method of claim 21, wherein the gelling composition comprises at least 1 w/w %, preferably at least 2 w/w % based on the total weight of the composition, of the cryoscopic agent.

23. Method of claim 21 or 22, wherein the gelling composition comprises 10 or less w/w %, preferably 7 or less w/w %, based on the total weight of the composition, of the cryoscopic agent.

24. Method of any of the claims 21-23, wherein the cryscopic agent comprises a mono- or disaccharide or a combination of two or more thereof.

25. Method of claim 24, wherein the mono- of disaccharide is chosen from the group, consisting of glucose, fructose, sucrose, maltose and lactose or a combination of two or more thereof.

26. Method of any of the claims 21-25, wherein the cryoscopic agent comprises fructose.

27. Method of any of the preceding claims, wherein the gelling composition comprises peptides having a molecular weight of up to 20 kDa, preferably up to 5 kDa.

28. Method of claim 27, wherein the peptides are derived from hydrolysed gelatin.

29. Method of claim 27 or 28, wherein the molecular weight of the peptides is between 100 and 5000 Da, preferably between 500 and 5000 Da.

30. Method of any of claims 27-28, wherein the gelling composition comprises at least 5 w/w %, based on the total weight of the composition, peptides, preferably hydrolyzed gelatin.

31. Method of any of the claims 27-30, wherein the gelling composition comprises 15 or less w/w %, based on the total weight of the composition, peptides, preferably hydrolyzed gelatin.

32. Method of any of the preceding claims, wherein the gelling composition comprises an anti-foaming agent.

33. Method of claim 32, wherein the anti-foaming agent comprises hydrophilic C8-C10 mono-, di- en triglycerides.

34. Method of claim 32 or 33, wherein the gelling composition comprises 10-1000 ppm anti-foaming agent.

35. Method of any of the preceding claims, wherein the gelling composition comprises one or more additional substances, chosen from the group, consisting of food supplements, such as nutrients, vitamins, calcium, trace elements; flavouring agents such as taste improvers; odorants; colorants; pharmaceuticals.

36. Method of any of the claims 1-35, further comprising providing a second gel layer onto the foodstuff.

37. Method of claim 36, wherein the composition of the second gel layer differs from that of the first gel layer.

38. Method of claim 37, wherein the second gel layer differs from the first in presence and/or composition of one or more additional substances.

39. Gelling composition comprising, based on the total weight of the composition:
12-25 w/w %, preferably 16-20 w/w % gellable hydrocolloids, 5-15 w/w %, preferably 7.5-12.5 w/w % peptides having a molecular weight of up to 20 kDa, 2-12 w/w %, preferably 4-8 w/w % plasticizer, 1-10 w/w %, preferably 3-7 w/w % cryoscopic agent, and optionally 10-1000 ppm, preferably 20-40 ppm anti-foaming agent, 0.5-15 w/w %, preferably 2-4 w/w % additional substances.

40. Gelling composition of claim 39, wherein the gellable hydrocolloid comprises gelatin.

41. Gelling composition of claim 40, wherein the gelatin has a bloom value of 100-300.

42. Gelling composition of any of claims 39-41, wherein the plasticizer is comprises a polyol.

43. Gelling composition of claim 42, wherein the plasticizer comprises glycerol or sorbitol or a combination thereof.

44. Gelling composition of claim 43, wherein the plasticizer comprises glycerol.

45. Gelling composition of any of claims 39-44, wherein the said peptides have a molecular weight of up to 5 kDa, preferably between 100 and 5000 Da, more preferably between 500 and 5000 Da.

46. Gelling composition of claim 45, wherein the peptides are derived from hydrolysed gelatin.

47. Gelling composition of any of claims 39-46, wherein the cryscopic agent comprises a mono- or disaccharide or a combination of two or more thereof.

48. Gelling composition of claim 47, wherein the mono- of disaccharide is chosen from the group, consisting of glucose, fructose, sucrose, maltose and lactose or a combination of two or more thereof.

49. Gelling composition of claim 48, wherein the cryoscopic agent comprises fructose.

50. Gelling composition of any of claims 39-49, wherein the gelling composition comprises one or more additional substances, chosen from the group, consisting of food supplements, such as nutrients, vitamins, calcium, trace elements, neutraceuticals, antioxidants; flavouring agents such as taste improvers; odorants; colorants; and pharmaceuticals.

51. Foodstuff, comprising, adhered to the outer surface thereof, a gel layer of a gelling composition of any of the claims 39-50.

52. Foodstuff of claim 51, being a pre-prepared meal.

53. Foodstuff of claim 51 or 52, the foodstuff being stored until consumption under freezing conditions, under refrigerator conditions or at ambient temperature, and intended to be heated for consumption to at least 50° C.

54. Foodstuff of any of claims 51-53, wherein the gel layer is a continuous gel layer.

55. Foodstuff of any of claims 51-54, wherein the gel layer has a thickness of 0.4 mm-1.6 mm, preferably of 0.6-1.2 mm.

56. Foodstuff of any of claims 51-55, wherein the gelling composition gels at ambient temperature.

57. Foodstuff of any of claims 51-56, wherein the gelling composition liquefies at a temperature above 40° C., preferably above 50° C., more preferably above 60° C.

58. Foodstuff of any of claims 51-57, wherein the gelling composition liquefies at a temperature below 90° C., preferably below 80° C., more preferably below 70° C.

59. Method for the preparation of a coated food stuff, comprising the application of the gelling composition of any of claims 1-50 onto the food stuff.

60. Method for the preparation of a liquid gelling composition of any of claims 39-50, comprising the steps of:
a) providing an aqueous medium,
b) adding the plasticizer to the aqueous medium,
c) adding the gellable hydrocolloid to the aqueous medium,
d) mixing to a homogenous mixture,
e) allowing the mixture of step d) to swell,
f) melting the swollen composition of step e) providing the liquid gelling composition.

61. Medicament or food supplement, included in a gelling composition of any of claims 39-50.

62. Method for improving the therapy compliance of a patient or patient group, comprising providing a food stuff according to any of the claims 51-58 to the said patient or patient group, the gelling composition comprising one or more pharmaceuticals for the said therapy.

63. Method of claim 62, wherein the food stuff is heated before providing to the patient or patient group.

64. Method of administration one or more of food supplements, such as nutrients, vitamins, calcium, trace elements, neutraceuticals, antioxidants, and/or pharmaceuticals to a subject, comprising the step of allowing the subject to ingest a food stuff according to any of the claims 51-58, the gelling composition comprising the one or more food supplements and/or pharmaceuticals.

65. Method of claim 64, wherein the foodstuff is heated before allowing the subject to ingest the said food stuff.
